(19) United States
(12) Patent Application Publication
(10) Pub. No.: US 2004/0029843 A1
(43) Pub. Date: Feb. 12, 2004

(54) Rapidly disintegrating formulations for treating or preventing mucositis

(75) Inventor: James Ronald Lawter, Yardley, PA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: OraPharma, Inc.

(21) Appl. No.: 10/601,259

(22) Filed: Jun. 20, 2003

Related U.S. Application Data
(60) Provisional application No. 60/390,068, filed on Jun. 20, 2002.
(60) Provisional application No. 60/407,730, filed on Sep. 3, 2002.

(51) Int. Cl. A61K 31/65

(52) U.S. Cl. 514/152

(57) ABSTRACT

Mucositis is treated and/or prevented by administrating to a patient a rapidly disintegrating solid dosage form comprising a tetracycline. The dosage form may contain another agent such as an NSAID, an inflammatory cytokine inhibitor, a mast cell inhibitor, an MMP inhibitor, an NO inhibitor, or a mixture thereof. The dosage forms may optionally also contain an antifungal agent to prevent fungal overgrowth due to reduction in the normal oral flora by the tetracycline. The tetracycline is preferably one that is poorly absorbed from the gastrointestinal tract. Such compositions have the advantage of treating the entire gastrointestinal tract since the active ingredient is not removed from the tract via absorption. Further, such compositions minimize systemic exposure and accompanying side effects. The compositions can be formulated as solid dosage forms comprising a tetracycline which disintegrates in an aqueous medium or saliva within in a short period, for example, two minutes. . The dosage form can be, for example, a hard, compressed tablet adapted to rapidly disintegrate in saliva or an aqueous vehicle or a table prepared by freeze-drying a solution or suspension of the active ingredients.

### CROSS REFERENCE TO RELATED APPLICATIONS

Priority is claimed to U. S. Provisional applications Serial No. 60/390,068, filed Jun. 20, 2002, and 60/407,730, filed Sep. 3, 2002, the teachings of which are incorporated herein.

### FIELD OF THE INVENTION

The present application relates generally to rapidly disintegrating solid dosage forms containing a tetracycline and optionally other agents that are useful for treating or preventing mucositis, when administered topically to the oral cavity.

### BACKGROUND OF THE INVENTION

Mucositis is a dose-limiting side effect of cancer therapy and bone marrow transplantation and is not adequately managed by current treatment (Sonis, 1993a, “Oral Complications,” in: Cancer Medicine, pp. 2381-2388, Holand et al.; Eds., Lea and Febiger, Philadelphia; Sonis, 1993b, “Oral Complications in Cancer Therapy,” In: Principles and Practice of Oncology, pp. 2385-2394, De Vitta et al., Eds., J. B. Lippincott, Philadelphia). Oral mucositis is found in almost 100% of patients receiving radiotherapy for head and neck tumors, in about 40% of patients receiving chemotherapy, and in about 90% of children with leukemia (Sonis, 1993b, supra). Complications related to oral mucositis, though varying in the different patient populations, generally include pain, poor oral intake with consequent dehydration and weight loss, and systemic infection with organisms originating in the oral cavity leading to septicemnia (Sonis, 1993b; U.S. Pat. No. 6,025,326 to Steinberg et al.). In addition to the oral cavity, mucositis may also affect other parts of the gastrointestinal tract.

A variety of approaches to the treatment of oral mucositis and associated oral infections have been tested with limited success. For example, the use of an allopurinol mouthwash, an oral sucralfate slurry, and pentoxifyline were reported in preliminary studies to result in a decrease in mucositis. Subsequent randomized and controlled studies, however, have failed to demonstrate any benefit from treatment with these agents (Loprinzi et al., 1995, Sem. Oncol. 22 Suppl. 3): 95-97; Epstein & Wong, 1994, Int. J. Radiation Oncology Biol. Phys. 28:693-698; Verdi et al., 1995, Oral Surg. Oral Med. Oral Pathol. Oral Radiol. Endod. 80:36-42).

Other therapies have been directed at decreasing oral flora and the extent of oral infections. Several studies have shown that the use of a vancomycin paste and antibiotic lozenges containing polymixin B, tobramycin and amphotericin B in patients undergoing myelosuppresive chemotherapy or radiation therapy can result in a decrease in oral mucositis and in the incidence of sepsis due to alpha hemolytic streptococci (Barker et al., 1995, J. Ped. Hem. Oncol. 17:151-155; Spijkervet et al., 1991, In: Irradiation Mucositis, Munksgaard Press, pp. 43-50).

Other methods of treating or preventing mucositis using a variety of formulations have been reported. U.S. Pat. No. 5,545,668 to Skubitz et al. describes formulations containing glutamine. U.S. Pat. No. 5,635,489 to Haley, U.S. Pat. No. 4,961,926 to Gabrilove, and U.S. Pat. No. 5,102,870 to Florine et al., describe treatments using formulations containing growth factors or stimulating factors. Mouthrinses containing antimicrobial peptides such as protegrin as the effective ingredient have also been described by U.S. Pat. No. 6,025,326 to Steinberg et. al. A triclosan formulation for treating mucositis was reported in U.S. Pat. No. 5,945,089 to Libin.

Rothwell and Spektor (Special Care in Dentistry, January-February 1990, pages 21-25) have shown that patients to whom an oral rinse containing tetracycline, diphenhydramine, nystatin, and hydrocortisone was administered developed less severe mucositis than patients receiving a control rinse. The tetracycline was unstable in solution with the other ingredients and was therefore administered in a separate solution.

WO 99/45910 by Sonis and Fey describes a method for treating and preventing mucositis by administering a non-steroidal anti-inflammatory drug (NSAID), an inflammatory cytokine inhibitor, or a mast cell inhibitor in combination with a second different therapeutic agent, which may be an NSAID, an inflammatory cytokine inhibitor, a mast cell inhibitor, a matrix metalloproteinase (MMP) inhibitor or a nitric oxide inhibitor. The MMP inhibitor can be a tetracycline. Most of the active ingredients have side effects if absorbed systemically at effective dosages. Only the compositions containing the tetracyclines appear to significantly reduce the symptoms of mucositis.

Despite the clear need for therapeutic agents to treat oral mucositis, none of the treatments provide significant long-term relief or decrease the severity or duration of mucositis. As a result, there is no standard treatment for oral mucositis.

WO 01/19362 by Lawter describes formulations that include, as an active ingredient, a tetracycline, which is poorly absorbed from the gastrointestinal tract.

There are times when a portable, convenient or easily stored formulation would be desirable. Bottles of a mouthrinse are bulky, consume much storage space, cannot be easily carried in a purse or shoulder bag, and may not be suitable for active ingredients that are not stable in suitable liquid vehicles. However, alternative formulations should be easily prepared for administration, or be administrable directly to the mouth. Moreover, it may in some cases be desirable to prolong the contact between the active ingredients and the mucosal tissue to which the formulation is applied, something not readily achieved using a mouthrinse.

As shown by studies conducted by Bellm et. al., Oral Oncology 37 (2001) 42-49, patients with mucositis, due to the painful lesion present in their mouths, do not tolerate solid dosage forms that slowly disintegrate when placed in the oral cavity.

It is therefore an object of the present invention to provide a rapidly disintegrating solid dosage form containing a tetracycline. for treating or preventing mucositis.

It is another object of the present invention to provide a method of making a rapidly disintegrating solid dosage form containing a tetracycline for treating or preventing mucositis.

It is therefore an object of the present invention to provide a rapidly disintegrating solid dosage form for treatment or prevention of mucositis, which is convenient, easy to transport, store and administer.

It is a further object of the present invention to provide a treatment that is safe, efficacious and easy for the patient to use.

### SUMMARY OF THE INVENTION

Mucositis is treated and/or prevented by administrating to a patient a formulation, comprising an effective amount of a tetracycline, which rapidly disintegrates or dissolves following administration to the mouth or, when added to a suitable liquid, rapidly disintegrates or dissolves to provide a liquid which may be administered as a mouthrinse or oral liquid. As used herein, “rapidly” generally means that the dosage form dissolves or disintegrates within a short time, for example about two minutes.

The tetracycline can be in the form of a pharmaceutically acceptable salt or the base form. The formulation may contain other agents such as a non-steroidal anti-inflammatory drug (NSAID), an inflammatory cytokine inhibitor, a mast cell inhibitor, an MMP inhibitor, an NO inhibitor, or a mixture thereof. The formulations can optionally also contain an antifungal agent to prevent fungal overgrowth due to reduction in the normal oral flora by the tetracycline or another agent.

The formulation preferably provides for longer-term contact with the oral mucosa than a simple mouth wash. The tetracycline may readily dissolve in water, or have low water solubility. The tetracycline may also be readily absorbed or poorly absorbed from the gastrointestinal tract.

Preferably, the formulation comprises a poorly absorbed tetracycline, which has the advantage of treating the entire gastro-intestinal tract since the active ingredient is not removed from the tract via absorption. Further, such a formulation minimizes systemic exposure and accompanying side effects.

The formulation can be administered as a liquid or a solid dosage form. The liquid formulation can be in the form of a solution or suspension in a pharmaceutically acceptable carrier that is prepared by adding a rapidly disintegrating solid dosage form to a suitable liquid vehicle. The solid dosage form can be in the form of, for example, sugar-coated tablets, film-coated tablets, multiple compressed tablets (including layered and press coated tablets), tablets for making a solution, effervescent tablets, sustained release tablets, extruded tablets, frozen tablets, hard tablets, soft tablets, fast disintegrating tablets, pellets, granules, microspheres, powder or shaped powders.

The solid dosage form may form a solution or suspension upon contact with an aqueous medium. The dosage form includes a tetracycline, and a water-soluble or water dispersible carrier, which disintegrates in an aqueous medium within about two minutes. A buffer may be included to adjust the pH, for example, to about 4 to 8. The aqueous medium can be saliva or water in a volume of, for example, 10 ml, in which the solid dosage form disintegrates or dissolves to form a mouth rinse.

The formulation can be a solid hard, compressed tablet that rapidly disintegrates upon contact with an aqueous medium. The hard, compressed tablet includes a tetracycline, optionally additional agents, and a matrix including a non-direct compression filler and a lubricant. The dosage form can rapidly disintegrate or dissolve in the mouth of a patient and thereby liberate the tetracycline. The tablet has a friability of, for example, about 2% or less when tested according to the USP friability test method. The tablet has a hardness of at least about 15 Newtons or higher.

The solid dosage form may include a polyvalent metal ion complex of a tetracycline. The dosage forms described herein can be prepared by any process suitable for making the different dosage forms described herein. For example, the dosage form described herein can be prepared by a process that includes the following steps: (i) preparing a solution or suspension comprising a water-soluble or water dispersible carrier, a filler, and the tetracycline, a part of which may be present as a suspension of solid particles; (ii) forming discrete units of the solution or suspension; and (iii) removing the solvent from the discrete units under vacuum thereby forming solid dosage forms comprising a network of carrier/filler carrying a dose of the tetracycline. The tetracycline may be in a salt, base form or polyvalent metal ion complex.

As another example, the dosage form can be prepared by a process that includes the steps of: (i) preparing a mixture comprising water, a water-soluble or water dispersible carrier, a filler, and the tetracycline in the form of a polyvalent metal complex; (ii) forming discrete units of the mixture; and (iii) removing the solvent from the discrete units under vacuum thereby forming solid dosage forms comprising a network of carrier/filler carrying a dose of the tetracycline.

The dosage forms described herein can be used to treat or prevent mucositis such as oral mucositis resulting from radiation or chemotherapy for cancer by administering to a patient an effective amount of a solution or suspension formed by placing the solid dosage form described herein in an aqueous solution. The solution or suspension is administered as, for example, a mouth-rinse. Alternatively, a solid dosage form can be administered to the oral cavity of a patient, for example, sublingually, wherein the tetracycline is released.

### DETAILED DESCRIPTION OF THE INVENTION

I. Topical Formulations

Rapidly disintegrating solid dosage forms for topical administration to the oral cavity for treating mucositis have been developed. These include as the active ingredient to treat the mucositis a tetracycline, a carrier which may include a suspending agent or a pharmacologically acceptable solid such as sugar, gelatin, chitosan, or starch and include excipients modifying the viscosity, taste, stability, adherence or release properties, and optionally other active ingredients such as an anti-fungal agent. The tetracycline can be present as a pharmaceutically acceptable salt, a base form, or a polyvalent metal ion complex.

A. Tetracyclines

As used herein, tetracyclines include compounds that may or may not have antibiotic activity. The tetracyclines described herein can have high or poor water solubility and can be well absorbed or poorly absorbed from the gastrointestinal tract. Solubility may be reduced by forming poorly soluble salts. Preferred tetracyclines are those that are poorly absorbed when administered orally. Compounds which have bioavailibilities of 50% or less are considered to be poorly absorbed.

Tetracyclines are known to have pharmacological activities such as matrix metalloproteinase, nitric oxide synthetase or caspase inhibition that are independent of their antibiotic properties. These activities may be important in the treatment and prevention of mucositis. It is known that these pharmacological activities may be associated with tetracyclines that do not have significant antibiotic properties.

Tetracyclines are defined by the following structure:

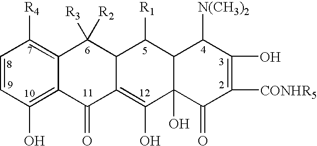

wherein R₁-R₅ may be a hydrogen atom, a halogen atom, a hydroxyl group, or any other organic composition comprising from 1-8 carbon atoms and optionally include a heteroatom such as nitrogen, oxygen, in linear, branched, or cyclic structural formats.

A wide range and diversity of embodiments within the definition of the above structure as are described within Essentials of Medicinal Chemistry John Wiley and Sons, Inc., 1976, pages 512-517. Preferably R₁ and R₂ are hydrogen or a hydroxyl group; R₃ is hydrogen or a methyl group; R₄ is a hydrogen atom, a halogen, or a nitrogen containing entity; and R₅ is a hydrogen atom, or nitrogen containing ring structure. The commonly known tetracycline analogues and derivatives include the following: oxytetracycline; chlortetracycline; demeclocycline; doxycycline; minocycline; rolitetracycline; lymecycline; sancycline; tetracycline; methacycline; apicycline; clomocycline; guamecycline; meglucycline; mepyclcline; penimepicycline; pipacycline; etocycline, penimocycline, and meclocycline.

Tetracycline derivatives that can be used as described herein include tetracycline derivatives modified at positions 1 through 4 and 10 through 12, although these modifications may result in reduction in antibiotic properties, according to Mitscher, et al., J. Med. Chem. 21(5), 485-489 (1978). The configuration of the 4 carbon is important to the antibiotic properties of the tetracyclines. For the antibiotic tetracyclines, carbon 4 is in the S configuration. The 4-epimers of the tetracyclines, which have the R configuration at the 4 carbon, have significantly reduced antibiotic activity. Other such non-antibiotic tetracycline analogs include the 4-de(dimethylamino) derivatives of the tetracyclines listed in the above paragraph. Specific examples include: 6demethyl-6-deoxy-4-dedimethylaminotetracycline; 6-demethyl-6-deoxy-4dedimethylamino-7-dimethylaminotetracycline; 6-demethyl-6-deoxy-4dedimethylamino-7-chloro-tetracycline; 4-hydroxy-4dedimethylaminotetracycline; 6a-deoxy-5-hydroxy-4dedimethylaminotetracycline; 4-dedimethylamino-5-oxytetracycline, and 4dedimethylamino-11-hydroxy-12a-deoxytetracycline. Further examples of tetracyclines with reduced antibiotic activity include 6-α-benzylthiomethylenetetracycline, 6-fluoro-6-demethyltetracycline, and 11α-chlorotetracycline.

Other tetracycline related compounds that can be used as described herein are the 9-((substituted)amido)tetracyclines. The latter include the compounds described in U.S. Pat. Nos. 5,886,175, 5,284,963, 5,328,902, 5,386,041, 5,401,729, 5,420,272, and 5,430,162.

Preferred poorly absorbed tetracyclines include compounds of the following structure:

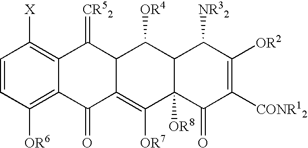

wherein R¹, R², R³, R⁴, R⁵, R⁶, R⁷, and R⁸ can be H, C1-C3 alkyl, phenyl, and aryl groups; and

wherein X is an H, alkyl, alkoxy, phenoxy, aryloxy, amino group, amide, acyl, and halo group; and pharmaceutically acceptable salts thereof.

The most preferred compound of this general structure is wherein R¹, R², R⁴, R⁵, R⁶, R⁷, and R⁸ are H;

wherein R³ is CH₃; and

wherein X is a chloro group. The generic name for this compound is meclocycline.

The preparation of meclocycline and its analogs and derivatives are known. For example, U.S. Pat. No. 3,966,808 to Luciano discloses methods for manufacturing 6-methylenetetracyclines.

B. Other agents

Active agents other than tetracycline can also be used in the formulation to aid in the treatment or prevention of mucositis. These agents can be inflammatory cytokine inhibitors, and/or mast cell inhibitors and/or NO inhibitors that reduce and inhibit mucositis.

As disclosed in PCT/US99/05437, proliferation of mast cells plays a key role in the development of mucositis. Mast cells are granule-containing secretory cells which are present in mucosal and connective tissues, and which can migrate within these tissues. The distribution of mast cells in tissues generally relates to the potential of mast cell-derived mediators to influence cells in the immediate environment. In the oral cavity, mast cells are preferentially distributed within the microvascular bed of the mucosa.

The granules of mast cells contain mediators that promote inflammation. Following degranulation, which can be triggered by a variety of stimuli, such as IgE, neuropeptides, trauma, and drugs, the mast cell mediators are deposited in large quantities in the extracellular environment. These mediators include histamine; the serine proteases chymase and tryptase; and cytokines, including TNF-alpha. The mediators promote inflammation by exerting their effects on endothelial cells and other cell types. For example, the mediators may influence adhesion molecules and the behavior of the tissue, leading to ulceration.

Two of the most important of these mediators are histamine and TNF-alpha. In the normal oral mucosa, these mediators are present only in the granules of mast cells, and are absent in other cells. Mast cell-released histamine increases vascular permeability by effecting structural changes, such as endothelial contraction and intercellular gap formation. These changes result in increased local levels of chemotherapy-induced damage. In addition, histamine promotes leukocyte adhesion to endothelial cells via transient mobilization of the adhesion molecule, P-selectin, thereby causing inflammation.

Another important mediator released by mast cells is the cytokine TNF-alpha. TNF-alpha contributes to the inflammatory process by releasing histamine and by inducing endothelial expression of E-selectin, an adhesion molecule that is critically required for the rapid adhesion of neutrophils, T cells, monocytes, and other leukocytes to endothelial cells.

Agents that inhibit the function of the mast cells or the action of the mediators released by mast cells can be used to treat and prevent mucositis. Mast cell inhibitors are chemical or biological agents that suppress or inhibit the function of mast cells, or the mediators released by mast cells. For example, mast cell inhibitors can inhibit degranulation, thereby preventing the release of mediators into the extracellular space.

Examples of mast cell degranulation inhibitors include picetannol, benzamidines, tenidap, tiacrilast, disodium cromoglycate, lodoxamide ethyl, and lodoxamide tromethamine. Other agents that inhibit mediator release include staurosporine and CGP 41251. Examples of mast cell mediator inhibitors include agents that block the release or secretion of histamine, such as FK-506 and quercetin; antihistamines such as diphenhydramine; and theophylline. Other mast cell inhibitors include serine protease inhibitors, such as alpha-1-protease inhibitor; metalloprotease inhibitors; lisofylline; TNFR-FE (available from Immunex, Seattle, Wash.); benzamidine; amiloride; and bis-amidines such as pentamidine and bis(5-amidino-2-benzimidazolyl)methane.

Inflammatory cytokine inhibitors are chemical or biological agents that suppress or inhibit inflammatory cytokines. Such inhibitors include pyridinyl imidazoles, bicyclic imidazoles, oxpentifylline, thalidomide and gabexate mesilate.

Anti-inflammatory agents can be used in combination with inflammatory cytokine and/or mast cell inhibitors to treat and prevent mucositis. Examples of anti-inflammatory agents that can be used include the non-steroidal anti-inflammatory drugs (NSAIDs) flurbiprofen, ibuprofen, ketoprofen, sulindac, and diclofenac. When NSAIDs are administered, anti-ulcer agents such as ebrotidine can be administered, e.g., to help protect against gastric mucosal damage. Other anti-inflammatory agents that can be used include misoprostil; methylxanthine derivatives, such as caffeine, lisofylline, or pentoxyfylline; benzydamine; naprosin; mediprin; and aspirin.

Another important class of anti-inflammatory agents includes cyclooxygenase (COX) inhibitors, particularly COX-2 inhibitors. COX-2, an inducible enzyme stimulated by growth factors, lipopolysaccharide, and cytokines during inflammation or cell injury, is responsible for the elevated production of prostaglandins during inflammation. COX-2 inhibitors are especially useful for treating mucositis in cancer patients undergoing chemotherapy or radiation therapy, because of the gastrointestinal tolerability of these inhibitors. COX-2 inhibitors that can be used include celecoxib, nimesulide, meloxicam, piroxicam, flosulide, etodolac, nabumetone, and 1-[(4methylsulfonyl)phenyl]-3-trifluoromethyl-5-[(4-fluoro)phenyl]pyrazole. Other useful anti-inflammatory agents include dual cyclooxygenase/lipoxygenase inhibitors, such as 2-acetylthiophene-2-thiazolylhydrazone, and leukotriene formation inhibitors, such as piriprost.

MMP inhibitors include both the antibacterial tetracyclines such as tetracycline HCI, minocycline and doxyocycline, as well as non-antibacterial tetracyclines.

Nitric oxide (NO) inhibitors can be any type. Preferable NO inhibitors can be aminoguanidine, guanidine or a mixture thereof.

The presence of bacteria in the oral cavity leads to secondary infection, serves as a source for systemic infection, and stimulates cytokine release, thereby amplifying tissue damage. The administration of anti-microbial agents in combination with the agents described above can result in an even more effective method for treating and preventing mucositis. Examples of antimicrobial agents that can be used include agents with activity against gram positive and gram negative organisms. Specific drugs include tetracycline HCI, amoxicillin, gentamicin, and chlorhexidine.

Other agents that may be used to treat or prevent mucositis include the nuclear transcription factor kappa-B (NF-B) activation inhibitors capsaicin and resiniferatoxin.

Other medicinal agents may be added for purposes of alleviating other undesirable conditions in the mouth. Such agents may include, for example, local anesthetics, antibacterial agents, and emollients, as well as anti-fungal agents.

Anti-Fungal Agents

Antibiotic tetracyclines applied topically in the oral cavity may reduce the number of susceptible flora to such an extent that competitive conditions that hold non-susceptible organisms in check may not be effective. In particular, fungi, which are not susceptible to tetracyclines, may increase drastically in number. To avoid this, an antifungal agent may be added to the composition. Examples of antifungal agents that have been shown to be effective in preventing or treating fungal overgrowth are nystatin and clotrimazole. The approved dosage for clotrimazole, 10 mg is three times a day for mucositis. The approved dosage of Nystatin is 200,000 to 400,000 units, 4 to 5 times a day for up to 14 days in pastilles.

Examples of local anesthetics are lidocaine and a eutectic mixture of lidocaine and prilocaine.

C. Pharmaceutically Acceptable Components

Vehicles for mouthrinse formulations

The rapidly disintegrating solid dosage forms contain preferably 0.1-50.0 mg, most preferably 1 to 10 mg, of the tetracycline. The solid dosage forms may be added to a liquid vehicle to produce a mouthrinse. The mouthrinse is preferably prepared by the patient immediately prior to administration.

The mouthrinse compositions are t administered to the oral cavity, held and swished around in the mouth, and then swallowed or spit out . . . . The liquid vehicle is preferably water. Other components may be present in the vehicle as described below.

Tetracyclines in general may not be sufficiently stable in aqueous media to permit formulations with long shelf lives at room temperature, i.e. a year or more, to be prepared. Stability of the tetracyclines varies greatly with structure. However, solids for re-constitution as aqueous based liquids prepared either by the patient or by a pharmacist prior to administration to the patient can be used, even for the least stable members of the class. Also polyvalent metal ion complexes may be prepared that are stable in contact with water at room temperature for two years or more. Examples are the calcium and magnesium organic or inorganic salts or complexes. These salts or complexes may be suspensions in water.

The stability of the tetracyclines in aqueous solutions is pH dependent. Procedures for choosing the optimum pH and buffering agents are well known. Other factors that affect stability in solution are also well known. For example, antioxidants may be added to reduce the rate of degradation due to oxidation.

An aqueous liquid preparation may contain buffers, surfactants, humectants, preservatives, flavorings, stabilizers (including antioxidants), colorants, and other additives used in preparations administered into the oral cavity. The compositions used as mouthwashes preferably should have a pH of about 4 to 8. A preparation having a pH of less than about 4 would be likely to cause a stinging sensation. Furthermore, the preparations having a higher pH are often unpleasant to use. The active agents need not be in solution to be effective. The active agents may be present wholly or in part as suspensions in a pharmacologically acceptable carrier, for example, water.

Generally, a water solution of tetracycline has a pH in the weak acidic range, e.g., pH 2-6. The preparations are buffered as necessary to provide the appropriate pH range, for example pH about 4-8. Appropriate buffer systems include citrate, acetate, tromethamine and benzoate systems. Preferably, the buffer system is tromethamine, which has a pKa of in the range of pKa 8-9. However, any buffer system commonly used for preparing medicinal compositions would be appropriate. While the vehicle used generally is primarily water, other components may be present such as alcohols, glycols (polyethylene glycol or polypropylene glycol are examples), glycerin, and the like may be used to solubilize the active agents or increase viscosity. Surfactants may include anionic, nonionic, amphoteric and cationic surfactants, which are known in the art as appropriate ingredients for mouthwashes.

Liquid formulations may contain additional components to improve the effectiveness of the product. For example, component(s) may be added to increase viscosity to provide improved retention on the surfaces of the oral cavity. Suitable viscosity increasing agents include carboxyalkyl, hydroxyalkyl, and hydroxyalkyl alkyl celluloses, xanthan gum, carageenan, alginates, pectins, guar gum, polyvinylpyrolidone, gellan gums, and gelatin. High viscosity formulations may cause nausea in chemotherapy and radiation patients and are therefore not preferred. Gelatin or its derivatives are preferred as viscosity modifying agents. Gellan gums are also preferred modifying agents since aqueous solutions or suspensions containing certain gellan gums may be prepared so that they will experience an increase in viscosity upon contact with electrolytes. Saliva contains electrolytes that will interact with such a gellan containing solution so as to increase their viscosity. The increased viscosity will promote retention of the solutions in the oral cavity and provide greater effectiveness due to increased contact time with the affected tissues.

In order to improve the patient acceptability, it is desirable to add an appropriate coloring and/or flavoring material. Any pharmaceutically acceptable coloring or flavoring material may be used. Flavorings used in the mouth rinse art such as peppermint, citrus flavorings, berry flavorings, custard, vanilla, cinnamon, and sweeteners, either natural or artificial, may be used. Flavorings that are known to increase salivary electrolyte concentrations may be added to increase the magnitude of the viscosity change.

Additional antimicrobial may be necessary to inhibit microbial growth. Suitable preservatives include alkyl parabens, benzoic acid, and benzyl alcohol. The quantity of preservative may be determined by conducting standard antimicrobial preservative effectiveness tests such as that described in the United States Pharmacopoeia.

Components for solid dosage form

Pharmaceutically acceptable fillers and excipients can be used to formulate the tetracyclines and the other optional agents described herein into solid dosage forms. Suitable solid dosage forms include powders or tablets that are designed for constitution as solutions by dissolution or suspension in a liquid vehicle . . . . In one preferred embodiment, the solid dosage form is a tablet.

For convenience of use, solid dosage forms designed to be used to prepare a liquid dosage form prior to administration preferably are rapidly disintegrating. Technologies to produce rapidly disintegrating solids are well known in the art. These include spray-drying, freeze-drying, particle size reduction and optimizing the pH of the dissolution medium.

Additional excipients generally known in the art can be used to formulate the tetracyclines and optional agents into a suitable dosage form (see, for example, Encyclopedia of Controlled Drug Delivery, Edith Mathiowitz, Ed., John Wiley & Sons, Inc., New York, 1999; and U.S. Pat. No. 5,558,880, the teachings of which and references cited therein are incorporated herewith by reference). For example, for a solid dosage form such as tablet prepared by a freeze-drying process, sugars such as lactose and/or mannitol or the derivatives thereof can be used in the formulation.

The solubilities of tetracyclines are a function of pH since they have several ionizable functional groups. Tetracyclines generally have a minimum in their pH-solubility curves between a pH of 3 and 6. The rate of dissolution of acidic salts may be increased by dissolving in a neutral to basic buffer. Dispersal of such salts may optimally be done at low pH.

Various solid dosage forms, the materials making the solid dosage forms, and methods for making the solid dosage forms have been described. For example, U.S. Pat. Nos. 6,316,027; 5,648,093; and 4,754,597 disclose fast disintegrating dosage forms of a drug and the process of making the dosage forms. U.S. Pat. Nos. 6,156,339; 5,837,287; 5,827,541 describe methods for the preparation of solid rapidly disintegrating dosage forms of a drug. Various forms of blister pack and the method of making the pack or the blister pack form of a drug has been described in, for example, U.S. Pat. Nos. 5,729,958; 5,046,618; 5,343,672; and 5,358,118. U.S. Pat. No. 5,631,023 discloses rapidly dispersing pharmaceutical tablets of a drug. U.S. Pat. No. 5,558,880 discloses a fast disintegrating, solid dosage form formed of a matrix containing gelatin, pectin and/or soy fiber protein. U.S. Pat. No. 5,188,825 describes using an ion exchange resin to bond a water soluble active agent so as to form a substantially water insoluble complex. The teachings of these U. S. patents are incorporated herein by reference.

In one embodiment, the tetracycline is formulated into a solid dosage form that forms a solution or suspension upon contact with an aqueous medium. The dosage form includes a tetracycline and, optionally, the other agents described herein and a buffer which disintegrates in the aqueous medium within two minutes to form a solution with a pH greater than 5. In one embodiment, the aqueous medium is saliva. In another embodiment, the aqueous medium is water in a volume of, for example, 10 ml, in which the solid dosage form rapidly disintegrates to form a mouth rinse in situ.

In another embodiment, the solid dosage form is a hard, compressed dosage form such as tablet that rapidly disintegrates upon contact with an aqueous medium. The hard, compressed dosage includes a tetracycline and, optionally the other agents, described herein, and a matrix including non-direct compression filler and a lubricant. The dosage form is adapted to rapidly disintegrates in the mouth of a patient and thereby liberate the tetracycline. The hard, compressed dosage has a friability of, for example, about 2% or less when tested according to the USP friability test method. The dosage form has a hardness of at least about 15 Newtons or higher. Hard, compressed dosage forms have been described, for example, in U.S. Pat. Nos. 6,221,392; 6,024,981; and 5,576,014, the teachings of which have been fully incorporated herein by reference.

In still another embodiment, the formulation described herein is a solid dosage form that includes a tetracycline and, optionally, the other agents described herein, which disintegrates within a short period, preferably two minutes, when placed in an aqueous medium to form a suspension which releases the tetracycline and the optional agent. The aqueous medium can be saliva or water.

In still another embodiment, the formulation described herein is a solid dosage form that includes a polyvalent metal ion complex of a tetracycline andoptional agent. The dosage form disintegrates within a short period, preferably, two minutes, when placed in an aqueous medium to form a suspension or paste comprising the tetracycline and/or antiinflammatory agent. The aqueous medium can be saliva or water. Preferably, the tetracycline and optional agent are released over a period of two minutes or longer when placed in the aqueous medium.

In still another embodiment, the formulation described herein is a solid pharmaceutical dosage form that includes a tetracycline and optional agent and a water-soluble or water dispersible carrier adapted for dissolution in the oral cavity over a period of more than two minutes. This can be done by incorporating components in the solid dosage form, which will disperse in water and retain the tetracycline. Such components are chitosan and gelatins, which have isoelectric points of about 7 or higher. These components will be positively charged and bind the negatively charged tetracycline at salivary pH.

The solid formulation can be in any dosage form. Representative dosage forms include, but are not limited to, sugar-coated tablets, film-coated tablets, multiple compressed tablets (including layered and press coated tablets), tablets for solution, effervescent tablets, sustained release tablets, extruded tablets, frozen tablets, , pills, pellets, granules, microspheres, powders and shaped powders.

II. Process of Preparing Rapidly Disintegrating Solid Tetracycline Dosage Forms

Various methods for making rapidly disintegrating solid dosage forms of a drug have been described in, for example, U.S. Pat. Nos. 6,316,027; 5,648,093; 4,754,597; 6,156,339; 5,837,287; 5,827,541; 5,729,958; 5,046,618; 5,343,672; 5,358,118; 5,631,023; 5,558,880; 5,188,825; 6,221,392; 6,024,981; and 5,576,014, the teachings of which are incorporated herein by reference.

The preparation of solid dosage forms varies with the particular form of the solid dosage. In one embodiment, the process involves the following steps: (i) preparing a solution of a water-soluble or water dispersible carrier, a filler, and a tetracycline and optionally additional agents; (ii) forming discrete units of the solution; and (iii) removing the solvent from the discrete units under vacuum thereby forming solid dosage forms comprising a network of carrier/filler delivering a dose of the tetracycline and the other agent.

In another embodiment, the process of making a solid dosage form involves: (i) preparing a suspension comprising water, a water-soluble or water dispersible carrier, a filler, and a tetracycline a part of which is present as a suspension of solid particles; (ii) forming discrete units of the suspension and (iii) removing the solvent from the discrete units under vacuum thereby forming solid dosage forms comprising a network of carrier/filler delivering a dose of the tetracycline.Optionally, additional agents may be added to the suspension and may dissolve completely or may be present as a suspension.

In still another embodiment, the process of making a solid dosage form involves: (i) preparing a mixture comprising water, a water-soluble or water dispersible carrier, a filler, and a tetracycline in the form of a polyvalent metal ion complex; (ii) forming discrete units of the mixture; and (iii) removing the solvent from the discrete units under vacuum thereby forming solid dosage forms comprising a network of carrier/filler delivering a dose of the tetracycline. Optionally, additional agents may be added to the mixture and may dissolve completely or may be present as a suspension.

III. Methods of Treatment

Methods of using the formulations disclosed herein generally involve applying the formulations topically to mucosal surfaces of the oral cavity and gastro-intestinal tract. In the preferred embodiment, one to eight applications per day beginning 24 hours before chemotherapy or radiation until conclusion of treatment are made. The typical volume of a mouthwash would be between 5-15 ml, preferably about 10.0 ml.

Therapy may continue for as long as the patient is receiving radiation or chemotherapy.

In one embodiment, the method is for treating or preventing oral mucositis resulting from radiation or chemotherapy for cancer. The method includes the step of administering to a patient an effective amount of a solution or suspension formed by placing one of the solid dosage forms containing a tetracycline in an aqueous solution. The solution is administered as, for example, a mouth-rinse. Optionally, additional agents may be present in the solid dosage form.

In another embodiment, the method for treating or preventing oral mucositis resulting from radiation or chemotherapy for cancer includes the step of administering a solid dosage form described herein to the oral cavity of a patient, for example, sublingually, wherein active agent are released.

The method disclosed herein can be further understood by reference to the following non-limiting examples.

Methods and Materials

The following animal model was used to demonstrate the effectiveness of the poorly absorbed tetracyclines in treating mucositis.

Hamsters were randomly assigned to treatment groups with eight (8) animals per group. Each group was treated either with a drug solution or a control, water.

Animals were dosed three times a day for 22 days. The first dose was applied on day -1. Either a solution of the drug or water alone was applied in a volume of 0.1 ml three times per day.

Mucositis was induced by acute radiation exposure of the check pouch. A single dose of radiation (35 Gy/dose) was administered to all animals on Day 0. Prior to irradiation, animals were anesthetized with an intraperiotoneal injection of sodium pentobarbital (80 mg/kg) and the left buccal pouch was everted, fixed and isolated using a lead shield.

Beginning on day 6 and continuing every other day up to day 28, the cheek pouch was photographed. On days that photographs were taken, prior to the first dosing of the day, the animals were anesthetized using an inhalation anesthetic and the left cheek pouch of each animal was rinsed vigorously with sterile water to remove residual food debris or foreign contamination and blotted dry with a gauze sponge. The appearance of the cheek pouch was scored visually by comparison to a validated photographic scale, ranging from 0 for normal to 5 for severe ulceration (clinical scoring). In descriptive terms, this scale is defined as follows:

Score Description

0 Pouch completely healthy. No erythema or vasodilatation

1 Light to severe erythema and vasodilatation. No erosion of mucosa

2 Severe erythema and vasodilatation. Erosion of superficial aspects of mucosa leaving denuded areas. Decreased stippling of mucosa

3 Formation of off-white ulcers in one or more places. Ulcers may have a yellow/gray color due to pseudomembrane formation. Cumulative size of ulcers up to ¼ of the pouch surface. Severe erythema and vasodilatation

4 Cumulative size of ulcers ¼ to ½ of the pouch surface. Loss of pliability. Severe erythema and vasodilatation

5 Virtually all of pouch is ulcerated. Loss of pliability (pouch can only partially be extracted from mouth).

A score of 1-2 represents mild stage of the disease, whereas a score of 3-5 indicates moderate to severe mucositis.

### EXAMPLE 1.

### Treatment with Meclocycline Sulfosalicylate (0.1 mg/ml).

Eight hamsters were treated as described above with 0.1 ml of aqueous solutions containing 0.1 mg/ml meclocycline sulfosalicylate. The solution was prepared by dissolving meclocycline in an aqueous solution of a tromethamine buffer. Significantly lower scores were found in the group treated with the meclocycline solution than a group of hamsters treated with a placebo control consisting of the solution without meclocycline. Relative to the control group, the group treated with meclocycline had a reduction of more than 75% in the number of animal days with scores of 3 or more.

### EXAMPLE 2.

### Treatment with Tetracycline Hydrochloride (0.1 mg/ml).

Eight hamsters were treated as described above with 0.1 ml of aqueous solution containing 0.1 mg/ml tetracycline hydrochloride. Significantly lower scores were found in the group treated with the tetracycline solution than a group of hamsters treated with a placebo control consisting of the solution without tetracycline. Relative to the control group, the group treated with tetracycline had a reduction of more than 75% in the number of animal days with scores of 3 or more.

These examples demonstrate that the tetracycline compositions significantly reduce the severity of mucositis when administered topically to the oral mucosa. Further they show that meclocycline which is poorly absorbed is as effective as a well-absorbed tetracycline.

### EXAMPLE 3.

### Freeze-Dried Meclocycline Gellan Gum Formulations.

Meclocycline hydrochloride powder formed by freeze-drying in bulk is added to a solution containing gellan gum at a concentration of 0.5 mg/ml. The tetracycline concentration is 0.1 mg/ml. The solution also contains methyl and propyl parabens as antimicrobial preservatives at concentrations of 0.18% and 0.02%, respectively and tromethamine buffer.

### EXAMPLE 4.

### Miconized Meclocycline Gellan Gum Buffered Formulations.

Meclocycline hydrochloride powder formed by micronization is added to a solution containing gellan gum at a concentration of 0.5 mg/ml. The tetracycline concentration is 0.05 mg/ml. The solution also contains methyl and propyl parabens as antimicrobial preservatives at concentrations of 0.18% and 0.02%, respectively and tromethamine buffer.

### EXAMPLE 5.

### Spray-Dried Meclocycline Gellan Gum Formulation.

Meclocycline hydrochloride powder formed by spray drying is added to a solution containing gellan gum at a concentration of 0.5 mg/ml. The tetracycline concentration is 0.1 mg/ml. The solution also contains methyl and propyl parabens as antimicrobial preservatives at concentrations of 0.18% and 0.02%, respectively and tromethamine buffer.

### EXAMPLE 6.

### Micronized Meclocycline Buffered Formulation.

Meclocycline sulfosalicylate powder formed by micronization is added to water. The suspension is added to a second solution containing a tromethamine buffer to form a mixture with a pH of approximately 8.

### EXAMPLE 7.

### Meclocyline Coated Pellets.

Pellets comprised of an inner core of tromethamine buffer and a coating of meclocycline hydrochloride embedded in methyl cellulose is added to water to form a mouth rinse. The concentration of the tetracycline in the solution is 0.1 mg/ml.

### EXAMPLE 8.

### Meclocycline Tablets.

A rapidly disintegrating tablet containing meclocycline sulfosalicylate is added to water. The tablet disintegrates and a second tablet containing a buffer is added to the solution to raise the pH so that the tetracycline rapidly dissolves.

### EXAMPLE 9.

### Meclocycline Calcium Complex Suspension.

A meclocyline calcium complex suspension is formed by addition of the hydrochloride salt of meclocycline to a solution of calcium lactate, which has been made basic, by the addition of sodium hydroxide. The solution also contained methyl and propyl parabens as antimicrobial preservative and EDTA and sodium bisulfite as antioxidants. The solutions were sparged with nitrogen to remove dissolved oxygen prior to addition of the sodium bisulfite. The mixture is deaerated.

### EXAMPLE 10.

### Meclocycline Suspension.

A suspension of meclocycline sulfosalicylate is formed by addition of micronized drug to an aqueous solution containing 0.5% gellan gum and methyl and propyl parabens as antimicrobial preservative.

### EXAMPLE 11.

### Meclocycline Sulfosalicylate Suspension.

A suspension of meclocycline sulfosalicylate is formed by addition of micronized drug to a unit dose quantity of an aqueous solution containing 0.5% gellan gum. No antimicrobial preservative is required since the formulation is used immediately after preparation.

### EXAMPLE 12.

### Meclocycline Oral Rinse Solution.

A powder containing meclocycline hydrochloride and buffer to promote rapid dissolution is prepared by granulation. The powder is dissolved in water to form an oral rinse solution containing 0.05 mg/ml meclocycline.

### EXAMPLE 13.

### Effervescent Tablet Containing Meclocycline Formulation

An effervescent tablet containing meclocycline sulfosalicylate and sodium bicarbonate. The tablet is dissolved in water to form an oral rinse solution containing 0.1 mg/ml meclocycline.

Modifications and variations of the methods and materials described herein will be obvious to those skilled in the art and are intended to come within the appended claims.

### EXAMPLE 14.

### Meclocycline Base Tablet

A suspension of micronized meclocycline base in water containing mannitol and gelatin with an isoelectric point of 7.0 is dosed into depressions in an aluminum laminate sheet and freeze-dried to form tablets.

1. A solid dosage form comprising an effective amount of a tetracycline, wherein the solid dosage form rapidly disintegrates in an aqueous medium.

2. The solid dosage form of claim 1 wherein the aqueous medium is saliva.

3. The solid dosage form of claim 1 wherein the solution formed when said dosage form is placed in said aqueous medium is a mouth rinse.

4. The solid dosage form of claim 1 comprising a hard, compressed dosage form for direct oral dosing comprising
a matrix comprising a non-direct compression filler and a lubricant,
wherein the dosage form is adapted to rapidly dissolve in the mouth of a patient and thereby liberate the tetracycline,
wherein the dosage form has a friability of about 2% or less when tested according the USP friability test, and
wherein the dosage form has a hardness of at least about 15 Newtons.

5. The solid dosage form of claim 1 wherein the solid dosage form disintegrates within two minutes when placed in an aqueous medium to form a suspension or paste which slowly releases the tetracycline.

6. The solid dosage form of claim 5 wherein the tetracycline is released over a period of ten minutes or longer.

7. The solid dosage form of claim 1 comprising a polyvalent metal ion complex of an effective amount of tetracycline.

8. The solid dosage form of claim 1 prepared by:
(i) preparing a suspension comprising water, a water-soluble or water dispersible carrier, and the tetracycline, a part of which is present as a suspension of solid particles;
(ii) forming discrete units of the suspension; and
(iii) removing the solvent from the discrete units,
thereby forming solid dosage forms comprising a network of carrier containing tetracycline.

9. The solid dosage form of claim 8 wherein the tetracycline is in the form of a polyvalent metal complex.

10. The solid dosage form of claim 1 comprising a hard, compressed, rapidly dissolvable dosage form for direct oral dosing comprising a matrix comprising a non-direct compression filler and a lubricant, wherein the dosage form has a friability of about 2% or less when tested according the USP friability test, and wherein the dosage form has a hardness of at least about 15 Newtons.

11. The solid dosage form of claim 10 wherein the solid dosage form disintegrates within two minutes when placed in an aqueous medium to form a suspension or paste which slowly releases the tetracycline.

12. The solid dosage form of claim 1 wherein the tetracycline is in the base form.

13. The solid dosage form of claim 1 in the form of a tablet.

14. The solid dosage form of claim 1 prepared by:
(i) preparing a solution comprising water, a water-soluble or water dispersible carrier, and the tetracycline, a part of which is present as a suspension of solid particles;
(ii) forming discrete units of the suspension; and
(iii) removing the solvent from the discrete units, thereby forming solid dosage forms comprising a network of carrier containing tetracycline.

15. The solid dosage form of claim 1 further comprising an agent selected from the group consisting of an NSAID, an inflammatory cytokine inhibitor, a mast cell inhibitor, an MMP inhibitor, an NO inhibitor, an antifungal agent and mixtures thereof.

16. The solid dosage form of claim 1 selected from the group consisting of sugar-coated tablets, film-coated tablets, multiple compressed tablets including layered and press coated tablets, tablets for solution, effervescent tablets, sustained release tablets, extruded tablets, frozen tablets, hard tablets, soft tablets, pills, pellets, granules, microspheres, powder and shaped powders.

17. A process for preparing a dosage form as defined by claim 1 comprising an effective amount of a tetracycline which rapidly disintegrates in an aqueous medium, comprising:
(i) preparing a solution of a water-soluble or water dispersible carrier, a filler, and the tetracycline in the form of a polyvalent metal ion complex;
(ii) forming discrete units of the suspension or solution; and
(iii) removing the solvent from the discrete units whereby dosage forms are formed comprising a network of carrier/filler containing a dose of the tetracycline.

18. A method for treating or preventing oral mucositis resulting from radiation or chemotherapy for cancer comprising administering to a patient the dosage form of claim 1.
